(19) United States
(12) Patent Application Publication
(10) Pub. No.: US 2023/0201588 A1
(43) Pub. Date: Jun. 29, 2023

(54) ELECTRICAL STIMULATION METHOD FOR IMPEDANCE COMPENSATION AND NON-IMPLANTABLE ELECTRICAL STIMULATION SYSTEM

(71) Applicant: GIMER MEDICAL. Co. LTD., New Taipei City (TW)

(72) Inventors: Wan-Ting CHIANG, New Taipei City (TW); Jian-Hao PAN, New Taipei City (TW)

(73) Assignee: GIMER MEDICAL. Co. LTD., New Taipei City (TW)

(21) Appl. No.: 17/979,375

(22) Filed: Nov. 2, 2022

(30) Foreign Application Priority Data
Dec. 29, 2021 (CN) 202111636547.7

(51) Int. Cl. A61N 1/36; A61N 1/04

(52) U.S. Cl. CPC A61N 1/36034; A61N 1/0496

(57) ABSTRACT

An electrical stimulation method for impedance compensation is applied to a non-implantable electrical stimulation device that provides high-frequency electrical stimulation. The non-implantable electrical stimulation device includes an electrical stimulator and an electrode assembly. The electrical stimulator is detachably electrically connected to the electrode assembly. The electrical stimulation method for impedance compensation includes the following steps. A high-frequency environment is provided and the first impedance value of the electrode assembly is calculated according to at least one of the measured first resistance value, first capacitance value, or first inductance value of the electrode assembly. The high-frequency environment is provided and the second impedance value of the electrical stimulator is calculated according to at least one of the measured second resistance value, second capacitance value, or second inductance value of the electrical stimulator. The first impedance value, and the second impedance value are stored for calculating the tissue impedance value.

### CROSS REFERENCE TO RELATED APPLICATIONS

This Application claims priority of China Patent Application No. 202111636547.7, filed on Dec. 29, 2021, the entirety of which is incorporated by reference herein.

### BACKGROUND

### Technology Field

The disclosure relates to an electrical stimulation technology.

### Description of the Related Art

In recent years, dozens of therapeutic nerve electrical stimulation devices have been developed, and at least tens of thousands of people undergo electrical stimulation device implantation every year. Due to the development of precision manufacturing technology, the size of medical devices has been miniaturized and may be implanted inside the human body, for example, an implantable electrical stimulation device.

Traditionally, the impedance value of an electrical stimulation device has been measured and written into the firmware of the electrical stimulation device or the external control device. However, in some situations (i.e., when an electrical stimulation device is used in a high-frequency environment), the impedance value of the electrical stimulation device may change, so that the actual impedance value does not match the value stored in the firmware, resulting in inaccurate control of the parameters of the stimulation signal that is generated, and even affecting its curative effect.

### SUMMARY

An electrical stimulation method for impedance compensation and a non-implantable electrical stimulation system are provided by the embodiment of the disclosure to overcome the problems mentioned above.

An embodiment of the disclosure provides an electrical stimulation method for impedance compensation. The electrical stimulation method for impedance compensation is applied to a non-implantable electrical stimulation device for providing high-frequency electrical stimulation. The non-implantable electrical stimulation device includes an electrical stimulator and an electrode assembly. The electrical stimulator is detachably electrically connected to the electrode assembly. The electrical stimulation method for impedance compensation includes the following steps. A high-frequency environment is provided and the first impedance value of the electrode assembly is calculated according to at least one of the measured first resistance value, first capacitance value, or first inductance value of the electrode assembly. The high-frequency environment is provided and the second impedance value of the electrical stimulator is calculated according to at least one of the measured second resistance value, second capacitance value, or second inductance value of the electrical stimulator. The first impedance value and the second impedance value are stored for calculating the tissue impedance value.

An embodiment of the disclosure provides a non-implantable electrical stimulation system. The non-implantable electrical stimulation system is applied to a high-frequency electrical stimulation operation. The non-implantable electrical stimulation system includes an electrode assembly, an electrical stimulator, and an impedance compensation device. The electrical stimulator is detachably electrically connected to the electrode assembly. The impedance compensation device is configured to provide a high-frequency environment and calculate the first impedance value of the electrode assembly according to at least one of the measured first resistance value, the first capacitance value, or first inductance value of the electrode assembly. The impedance compensation device provides the high-frequency environment and calculates the second impedance value of the electrical stimulator according to at least one of the measured second resistance value, the second capacitance value, or second inductance value of the electrical stimulator. The first impedance value and the second impedance value are stored in the electrical stimulator for calculating the tissue impedance value.

Other aspects and features of the disclosure will become apparent to those with ordinary skill in the art upon review of the following descriptions of specific embodiments of the electrical stimulation method for impedance compensation and the non-implantable electrical stimulation system provided by the embodiment.

### BRIEF DESCRIPTION OF THE DRAWINGS

The disclosure can be more fully understood by reading the subsequent detailed description and examples with references made to the accompanying drawings, wherein:

FIG. 1A is a perspective view of a non-implantable electrical stimulation device according to an embodiment of the disclosure;

FIG. 1B is a perspective view of a non-implantable electrical stimulation device shown in FIG. 1A form another angle;

FIG. 1C is an exploded schematic view of a non-implantable electrical stimulation device shown in FIG. 1A;

FIG. 2 is a block diagram of a non-implantable electrical stimulation device according to an embodiment of the disclosure;

FIG. 3 is a waveform diagram of an electrical stimulation signal of a non-implantable electrical stimulation device according to an embodiment of the disclosure;

FIG. 4 is a schematic view of a non-implantable electrical stimulation device according to an embodiment of the disclosure;

FIG. 5 is block diagram of an impedance compensation device according to an embodiment of the disclosure;

FIG. 6 is a schematic view of an impedance compensation model according to an embodiment of the disclosure; and

FIG. 7 is a flowchart of an electrical stimulation method for impedance compensation according to an embodiment of the disclosure.

### DETAILED DESCRIPTION OF THE DISCLOSURE

The following description is of the contemplated mode of carrying out the disclosure. This description is made for the purpose of illustrating the general principles of the disclosure and should not be taken in a limiting sense. The scope of the disclosure is determined by reference to the appended claims.

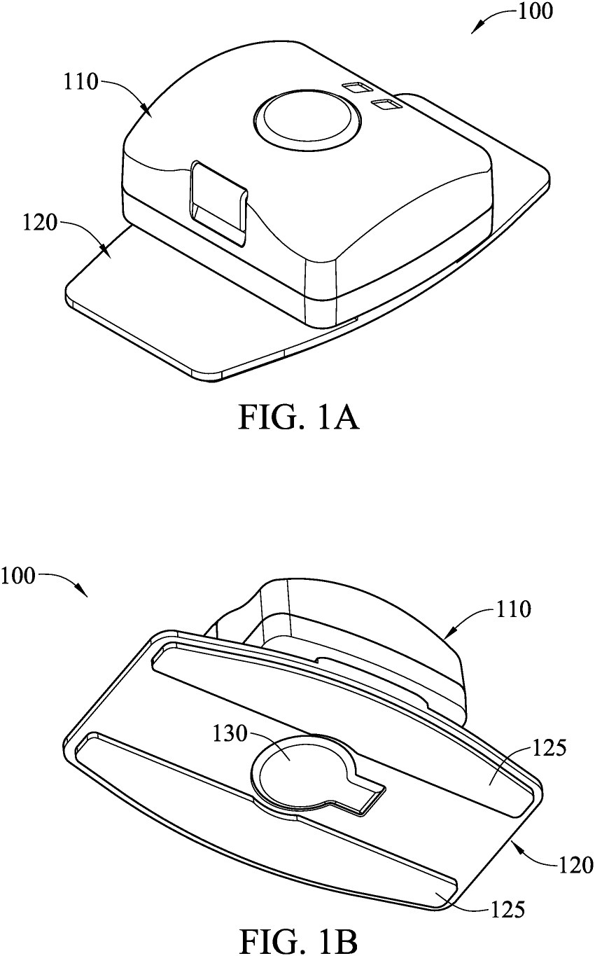

FIG. 1A is a perspective view of a non-implantable electrical stimulation device according to an embodiment of the disclosure. FIG. 1B is a perspective view of a non-implantable electrical stimulation device shown in FIG. 1A form another angle. FIG. 1C is an exploded schematic view of a non-implantable electrical stimulation device shown in FIG. 1A. Please refer to FIG. 1A, FIG. 1B and FIG. 1C. The non-implantable electrical stimulation device 100 includes an electrical stimulator 110 and an electrode assembly 120. In the embodiment, the non-implantable electrical stimulation 100 is, for example, a transcutaneous electrical nerve stimulation device (TENS device), which does not necessarily need to be implanted in the body or subcutaneously, but is directly attached to the body surface or skin of a living body through the electrode assembly 120 for electrical stimulation of a target area. In the embodiment, the living body is, for example, the body of a user or a patient. The target area includes the body surface or skin of the living body, and the target area is, for example, a superficial nerve within 10 millimeters (mm) from the body surface to relieve pain or other symptoms of disease. In addition, the main difference between the non-implantable electrical stimulation device 100 of the embodiment and the general muscle electrical stimulation device is that the target area for electrical stimulation performed by the non-implantable electrical stimulation device 100 of the embodiment is the nerve, rather than the muscle. Therefore, when the non-implantable electrical stimulation device 100 performs an electrical stimulation, the distance between the two electrodes of the electrode assembly 120 is relatively close, and the distance between the two adjacent electrodes is, for example, between 5 mm and 35 mm. The aforementioned two electrodes may be positive and negative electrodes, or one working electrode and another reference electrode, wherein the working electrode sends an electrical stimulation signal, and the reference electrode send a voltage signal at a DC fixed level.

In the embodiment, the electrical stimulator 110 is disposed on the upper half of the non-implantable electrical stimulation device 100. The electrical stimulator 110 includes a casing 111, a circuit board 112, at least two first electrical connectors 113 and at least one first magnetic unit 114.

The casing 111 includes an upper casing 111a and a lower casing 111b. The upper casing 111a and the lower casing 111b are combined to form an accommodating space. Most of the components required for the electrical stimulator 110 are disposed in the accommodating space, including the circuit board 112, the first electrical connectors 113, the first magnetic unit 114, and other components.

On the other hand, the electrode assembly 120 is disposed in the lower half of the non-implantable electrical stimulation device 100 and is connected with the lower casing 111b under of the electrical stimulator 110. The electrode assembly 120 includes a body 121, two electrodes 122, at least one second magnetic unit 123, at least two second electrical connectors 124 and a conductive gel 125. The electrical stimulator 110 may electrically transmit the sent electrical stimulation signal from the circuit board 112 to electrodes (i.e., the electrodes 122) of other components, such that the non-implantable electrical stimulation device 100 may perform electrical stimulation on the target area of the living body.

In the embodiment, the body 121 of the electrode assembly 120 has certain flexibility, such that it may be easily attached to different parts of the living body, and the material of the body 121 of the electrode assembly 120 may be rubber, silicone or other flexible materials.

In the embodiment, the electrode assembly 120 may be a magnetic electrode assembly. In addition, the above two electrodes 122 may be thin film electrodes. Furthermore, the above electrodes 122 are printed or sprayed on a surface F1 of the body 121 opposite to the casing 111 by a conductive material (i.e., silver paste), and the thickness of the above electrodes 122 may be 0.01 mm to 0.30 mm. The surface F1 is the lower surface of the body 121 shown in FIG. 1C, and is also a side facing the using part of the user during use.

In some embodiments, when using the non-implantable electrical stimulation device 100 of the embodiment, the conductive gel 125 of the electrode assembly 120 may be coated on the lower surface of the body 121. In some embodiments, the conductive gel 125 may be disposed on a sticking surface of the electrode 122 away from the body 121, and one electrode 122 may be correspondingly disposed with the conductive gel 125. The conductive gel 125 is not only sticky so that the electrode patch provided with the electrodes may be attached to the body surface or skin of the living body, but also reduces the contact resistance between the electrodes 122 and the body surface or skin of the living body due to the arrangement of the conductive gel 125, and may make the current of the electrodes evenly spread over the entire attached body surface area, avoiding the stinging sensation of the living body. At the same time, the comfort of using the non-implantable electrical stimulation device 100 is increased. That is, the electrode assembly 120 of the embodiment does not have a lead type, and the electrode assembly 120 may be two thin film electrodes 122 combined with the conductive gel 125 for electrical stimulation.

In addition, the first magnetic unit 114 of the electrical stimulator 110 is disposed in the accommodating space, for example, between the circuit board 112 and the casing 111. It should be noted that the first magnetic unit 114 in the embodiment is disposed under the circuit board 112.

In the non-implantable electrical stimulation device 100 of the embodiment, the electrical stimulator 110 includes at least one first magnetic unit 114, the electrode assembly 120 includes at least one second magnetic unit 123, and the numbers of the first magnetic unit 114 and the second magnetic unit 123 may be the same or different. The embodiment is described by taking as an example that four first magnetic units 114 correspond to four magnetic units 123. In addition, the electrode assembly 120 is detachably positioned on one side of the electrical stimulator 110 (i.e., one side of the lower casing 111b of the electrical stimulator 110) by being attracted by the at least one first magnetic unit 114 and the at least one second magnetic unit 123.

In addition, in the embodiment, the lower casing 111b of the electrical stimulator 110 may be correspondingly designed to have a protruding configuration 130 (as shown in FIG. 1B) at a position corresponding to the opening 126 of the body 121. After the electrode assembly 120 is assembled to the electrical stimulator 110, the protruding configuration 130 of the lower casing 111b protrudes from the opening 126 of the body 121. Therefore, the electrode assembly 120 may be more stably disposed on the electrical stimulator 110, and the alignment of the electrode assembly 120 and the electrical stimulator 110 is facilitated.

After the electrical stimulation signal is sent from the circuit board 112, the electrical stimulator 110 may be electrically connected to electrodes 122 through the first electrical connectors 113 and the second electrical connectors 124 (male rivets 124b and female rivets 124a) in sequence, and finally the electrical stimulation signal electrically stimulates the target area through the conductive gel 125 disposed corresponding to the electrodes 122. In the embodiment, in addition to above components, the non-implantable electrical stimulation device 100 is provided with a battery 115 or a power module in the accommodating space of the electrical stimulator 110, and the battery 115 or the power module may output power to the circuit board 112.

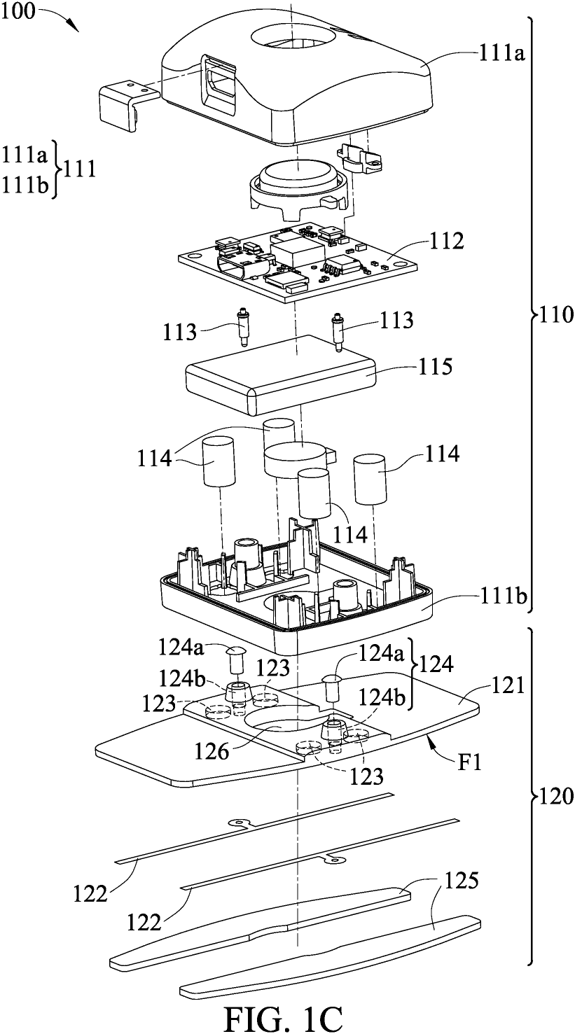

FIG. 2 is a block diagram of a non-implantable electrical stimulation device 100 according to an embodiment of the disclosure. As shown in FIG. 2, the non-implantable electrical stimulation device 100 may at least include a power management circuit 210, an electrical stimulation signal generating circuit 220, a measurement circuit 230, a control unit 240, a communication circuit 250 and a storage device 260. In addition, the electrical stimulation signal generating circuit 220, the measurement circuit 230, the control unit 240, the communication circuit 250 and the storage device 260 may be disposed on the circuit board 112 of the electrical stimulator 110 shown in FIG. 1C. It should be noted that the block diagram shown in FIG. 2 is only for the convenience of explaining the embodiment of the disclosure, but the disclosure is not limited to FIG. 2. The non-implantable electrical stimulation device 100 may also include other components.

According to an embodiment of the disclosure, the non-implantable electrical stimulation device 100 may be electrically coupled to an external control device 200. The external control device 200 may include an operation interface. According to the operation of the user on the operation interface, the external control device 200 may generate a command or a signal to be transmitted to the non-implantable electrical stimulation device 100, and transmit the command or the signal to the non-implantable electrical stimulation device 100 through a manner of a wired communication (i.e., a transmission line). According to an embodiment of the disclosure, the external control device 200 may be a smart phone, but the disclosure is not limited thereto.

Furthermore, according to another embodiment of the disclosure, the external control device 200 may also transmit the command or the signal to the non-implantable electrical stimulation device 100 through a manner of a wireless communication (i.e., Bluetooth, Wi-Fi, or NFC, but the disclosure is not limited thereto).

According to an embodiment of the disclosure, the non-implantable electrical stimulation device 100 and the external control device 200 may be integrated into one device. According to an embodiment of the disclosure, the non-implantable electrical stimulation device 100 may be an electrical stimulation device with the battery 115, or an electrical stimulation device with the power wirelessly transmitted by the external control device 200.

According to an embodiment of the disclosure, the power management circuit 210 is configured to provide the power to the internal components and circuits in the non-implantable electrical stimulation device 100. The power provided by the power management circuit 210 may be from a built-in rechargeable battery (i.e., the battery 115) or the external control device 200, but the disclosure is not limited thereto. The external control device 200 may provide the power to the power management circuit 210 through a wireless power supply technology. The power management circuit 210 may be activated or deactivated according to the command of the external control device 200. According to an embodiment of the disclosure, the power management circuit 210 may include a switch circuit (not shown). The switch circuit may be turned on or off according to the command of the external control device 200 to activate or deactivate the power management circuit 210.

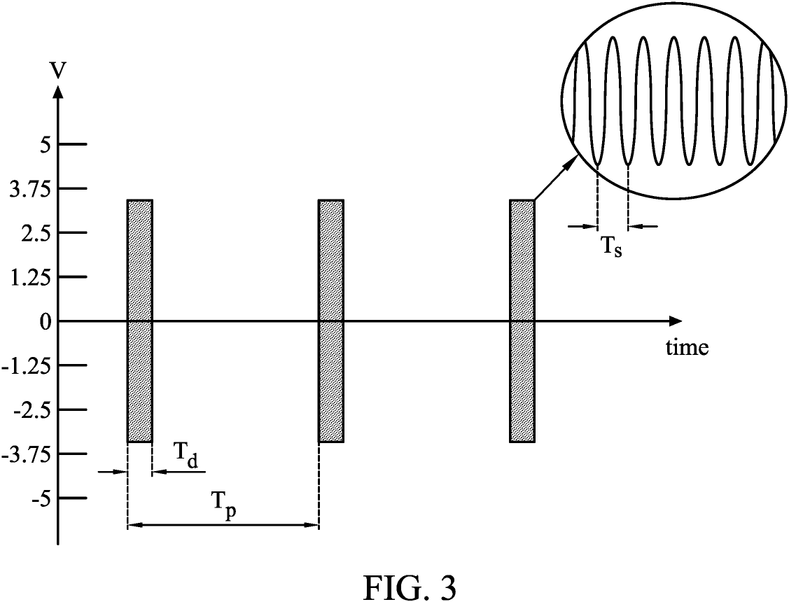

According to an embodiment of the disclosure, the electrical stimulation signal generating circuit 220 is configured to generate the electrical stimulation signal. The electrical stimulation signal generating circuit 220 may transmit the generated electrical stimulation signal to the electrodes 122 of the electrode assembly 122 through the first electrical connectors 113 and the second electrical connectors 124, so as to electrically stimulate the target area of the living body (i.e., a human or an animal) through the conductive gel 125 disposed corresponding to the electrodes 122. The above target area is, for example, a median nerve, a tibial nerve, a vagus nerve, a trigeminal nerve or other superficial nerves, but the disclosure is not limited thereto. The detailed structure of the electrical stimulation signal generating circuit 220 will be described with reference to FIG. 4.

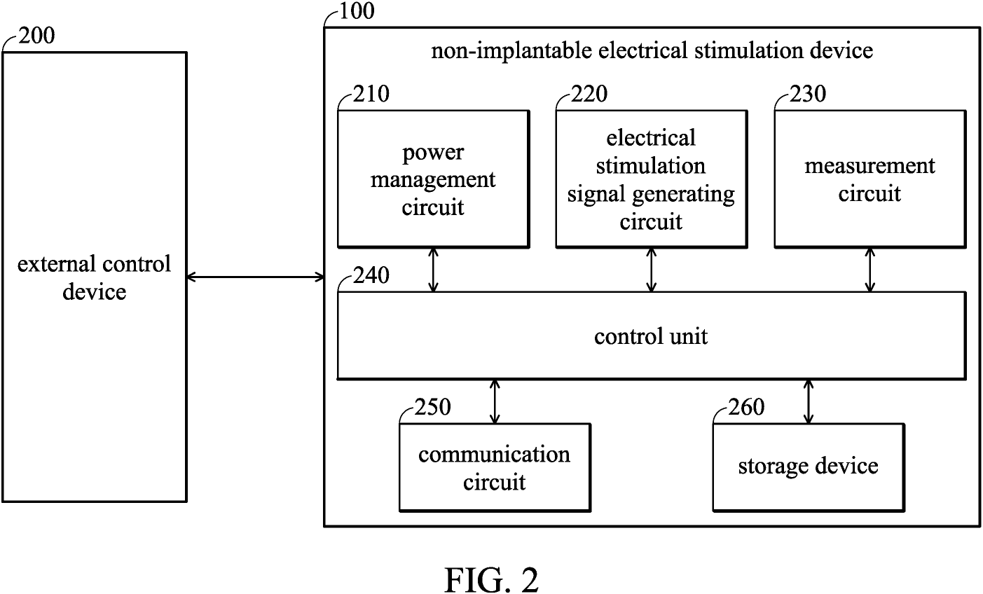

FIG. 3 is a waveform diagram of an electrical stimulation signal of a non-implantable electrical stimulation device according to an embodiment of the disclosure. As shown in FIG. 3, according to an embodiment of the disclosure, the above electrical stimulation signal may be a pulsed radio-frequency (PRF) signal (or referred to as a pulse signal), a continuous sine wave, a continuous triangular wave, etc., but the embodiment of the disclosure is not limited thereto. In addition, when the electrical stimulation signal is a pulse alternating signal, one pulse cycle time Tₚ includes a plurality of pulse signals and at least one rest period of time, and the pulse cycle time Tₚ is the reciprocal of the pulse repetition frequency. The pulse repetition frequency range (also referred to as the pulse frequency range) is, for example, between 0 and 1 KHz, preferably between 1 and 100 Hz. In the embodiment, the pulse repetition frequency of the electrical stimulation signal is, for example, 2 Hz. In addition, the duration time T_{d} of the plurality of pulses in one pulse cycle time is, for example, between 1 and 250 milliseconds (ms), preferably between 10 and 100 ms. In the embodiment, the duration time T_{d} is, for example, 25 ms. In the embodiment, the frequency of the electrical stimulation signal is 500 KHz, in other words, the cycle time Tₛ of the electrical stimulation signal is about 2 microseconds (µs). Furthermore, the frequency of the above electrical stimulation signal is the intra-pulse frequency in each pulse alternating signal of FIG. 3. In some embodiments, the above intra-pulse frequency range of the above electrical stimulation signal is, for example, 100 KHz to 1000 KHz. In some embodiments, the intra-pulse frequency range of the above electrical stimulation signal is, for example, from 200 KHz to 800 KHz. In some embodiments, the intra-pulse frequency range of the above electrical stimulation signal is, for example, from 480 KHz to 520 KHz. In some embodiments, the intra-pulse frequency of the above electrical stimulation signal is, for example, 500 KHz. The voltage range of the above electrical stimulation signal may be between -25 V and 25 V. Furthermore, the voltage range of the above electrical stimulation signal may further be between -20 V and 20 V. The current range of the above electrical stimulation signal may be between 0 and 60mA. Furthermore, the current range of the above electrical stimulation signal may further be between 0 and 50mA.

According to an embodiment of the disclosure, the user may operate the non-implantable electrical stimulation device 100 for electrical stimulation only when the user fells the need (for example, the symptoms become severe or not relieved). After the non-implantable electrical stimulation device 100 performs one electrical stimulation on the target area, the non-implantable electrical stimulation device 100 needs to wait a limited time before performing the next electrical stimulation on the target area. For example, after the non-implantable electrical stimulation device 100 performs one electrical stimulation on the target area, the non-implantable electrical stimulation device 100 needs to wait for 30 minutes (i.e., the limited time) before performing the next electrical stimulation on the target area, but the disclosure is not limited thereto. The limited time may also be any time interval within 45 minutes, 1 hour, 4 hours or 24 hours.

According to an embodiment of the disclosure, the measurement circuit 230 may measure the voltage value and the current value of the electrical stimulation signal according to the electrical stimulation signal generated by the electrical stimulation signal generating circuit 220. Furthermore, the measurement circuit 230 may measure the voltage value and the current value on the tissue of the target area of the living body (i.e., the body of the user or patient). According to an embodiment of the disclosure, the measurement circuit 230 may adjust the current and the voltage of the electrical stimulation signal according to the instruction of the control unit 240. The detailed structure of the measurement circuit 230 will be described with reference to FIG. 4.

According to an embodiment of the disclosure, the control unit 240 may be a controller, a microcontroller or a processor, but the disclosure is not limited thereto. The control unit 240 may be configured to control the electrical stimulation signal generating circuit 220 and the measurement circuit 230. The operation of the control unit 240 will be described below with reference to FIG. 4.

According to an embodiment of the disclosure, the communication circuit 250 may be configured to communicate with the external control device 200. The communication circuit 250 may transmit the command or the signal received from the external control device 200 to the control unit 240, and transmit the data measured by the non-implantable electrical stimulation device 100 to the external control device 200. According to an embodiment of the disclosure, the communication circuit 250 may communicate with the external control device 200 in a wireless or a wired communication manner.

According to an embodiment of the disclosure, when performing the electrical stimulation, all of the electrodes of the non-implantable electrical stimulation device 100 are activated or enabled. Therefore, the user does not need to select which electrodes on the electrode assembly 120 need to be activated, and which activated electrode is negative or positive polarity.

Compared with the traditional electrical stimulation, which is a pulse signal with a low frequency (i.e., 10 KHz), it is easy to cause the tingling sensation of the user or discomfort of the user due the paresthesia. In an embodiment of the disclosure, the electrical stimulation signal is a pulse signal with a high frequency (i.e., 500 KHz). Therefore, no paresthesia, or only a very slight paresthesia, may be caused.

According to an embodiment of the disclosure, the storage device 260 may be a volatile memory (i.e., random access memory (RAM)), a non-volatile memory (i.e., flash memory), a read only memory (ROM), a hard disk or a combination thereof. The storage device 260 may be configured to store files and data required for electrical stimulation. According to an embodiment of the disclosure, the storage device 260 may be configured to store the relevant information of a look-up table provided by the external control device 200.

FIG. 4 is a schematic view of a non-implantable electrical stimulation device 100 according to an embodiment of the disclosure. As shown in FIG. 4, the electrical stimulation signal generating circuit 220 may include a variable resistor 221, a waveform generator 222, a differential amplifier 223, a channel switch circuit 224, a first resistor 225 and a second resistor 226. The measurement circuit 230 may include a current measurement circuit 231 and a voltage measurement circuit 232. It should be noted that the schematic view shown in FIG. 4 is only for the convenience of explaining the embodiment of the disclosure, but the disclosure is not limited to FIG. 4. The non-implantable electrical stimulation device 100 may also include other components, or include other equivalent circuits.

As shown in FIG. 4, according to an embodiment of the disclosure, the variable resistor 221 may be coupled to a serial peripheral interface (SPI) (not shown) of the control unit 240. The control unit 240 may transmit the command to the variable resistor 221 through the serial peripheral interface to adjust the resistance value of the variable resistor 221, so as to adjust the magnitude of the electrical stimulation signal to be output. The waveform generator 222 may be coupled to a pulse width modulation (PWM) signal generator (not shown) of the control unit 240. The pulse width modulation signal generator may generate a square wave signal and transmit the square wave signal to the waveform generator 222. After the waveform generator 222 receives the square wave signal generated by the pulse width modulation signal generator, the waveform generator 222 may convert the square wave signal into a sine wave signal, and transmit the sine wave signal to the differential amplifier 223. The differential amplifier 223 may convert the sine wave signal into a differential signal (i.e., the output electrical stimulation signal), and transmit the differential signal to the channel switch circuit 224 through the first resistor 225 and the second resistor 226. The channel switch circuit 224 may sequentially transmit the differential signal (i.e., the output electrical stimulation signal) to the electrodes corresponding to each channel according to the instruction of the control unit 240.

As shown in FIG. 4, according to an embodiment of the disclosure, the current measurement circuit 231 and the voltage measurement circuit 232 may be coupled to the differential amplifier 223, so as to obtain the current value and the voltage value of the differential signal (i.e., the output electrical stimulation signal). In addition, the current measurement circuit 231 and the voltage measurement circuit 232 may be configured to measure the voltage value and the current value of the tissue of the target area of the living body (i.e., the body of the user or patient). Furthermore, the current measurement circuit 231 and the voltage measurement 232 may be coupled to an input/output (I/O) interface (not shown) of the control unit 240, so as to receive the instruction from the control unit 240. According to the instruction of the control unit 240, the current measurement circuit 231 and the voltage measurement 232 may adjust the current and the voltage of the electrical stimulation signal to the current value and the voltage value suitable for processing by the control unit 240. For example, if the voltage value measured by the voltage measurement circuit 232 is ±10 V and the voltage value suitable for processing by the control unit 240 is 0 \~3V, the voltage measurement circuit 232 may firstly reduce the voltage value to + 1.5 V and then raise the voltage value to 0 \~3V according to the instruction of the control unit 240.

After the current measurement circuit 231 and the voltage measurement circuit 232 adjust the current value and the voltage value, the current measurement circuit 231 and the voltage measurement circuit 232 may transmit the adjusted electrical stimulation signal to an analog-to-digital convertor (ADC) (not shown) of the control unit 240. The analog-to-digital convertor may sample the electrical stimulation signal to provide the control unit 240 for subsequent calculation and analysis.

According to an embodiment of the disclosure, when the electrical stimulation is to be performed on a target area of the body of a patient, the user (which may be a medical staff or the patient himself) may select an electrical stimulation lever from a plurality of electrical stimulation levels on the operation interface of the external control device 200. In an embodiment of the disclosure, different electrical stimulation levels may correspond to different target energy values. The target energy value may be a set of preset energy values. When the user selects an electrical stimulation level, the non-implantable electrical stimulation device 100 may acquire how many millijoules of energy to provide to the target area for electrical stimulation according to the target energy value corresponding to the electrical stimulation level selected by the user. According to an embodiment of the disclosure, during a trial phase, the plurality of target energy values corresponding to the plurality of electrical stimulation levels may be regarded as a first group of preset target energy values. According to an embodiment of the disclosure, the first group of preset target energy values (i.e., the plurality of target energy values) may be a linear sequence, an arithmetic sequence, or a proportional sequence, but the disclosure is not limited thereto.

According to an embodiment of the disclosure, before the non-implantable electrical stimulation device 100 performs the electrical stimulation on the target area, such as in the non-electrical stimulation state, the non-implantable electrical stimulation device 100 may calculate the tissue impedance value of the target area, and the obtained tissue impedance value may then be used to calculate the energy value of the electrical stimulation signal transmitted to the target area. According to an embodiment of the disclosure, such as non-implantable electrical stimulation device 100 shown in FIG. 1A, FIG. 1B and FIG. 1C, the non-implantable electrical stimulation device 100 may calculate the tissue impedance value according to an impedance value of the electrode assembly 120 and an impedance of the electrical stimulator 110. There will be a more detailed description below.

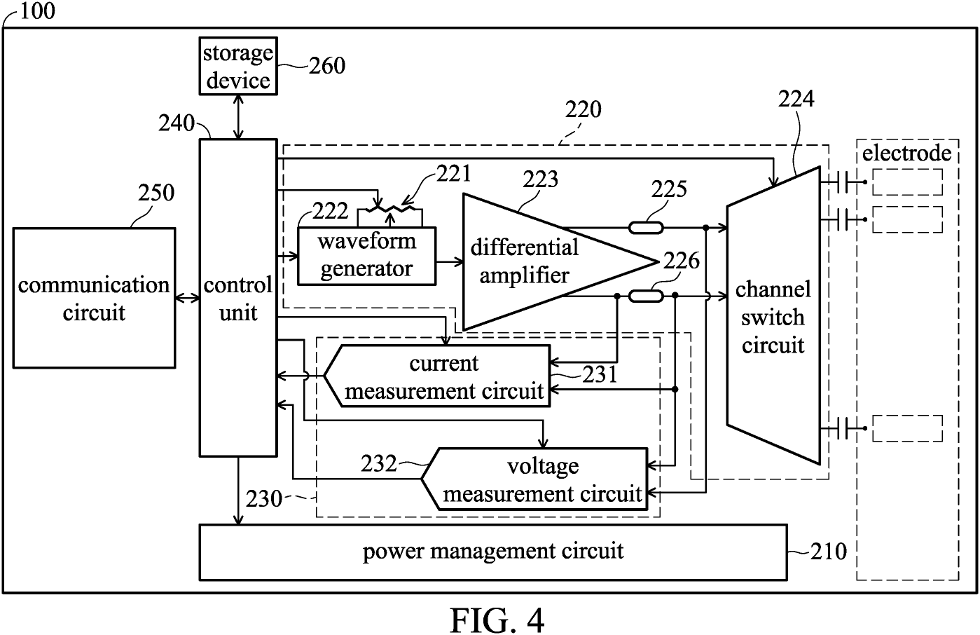

FIG. 5 is block diagram of an impedance compensation device 500 according to an embodiment of the disclosure. As shown in FIG. 5, the impedance compensation device 500 may include a measurement circuit 510, but the disclosure is not limited thereto. The measurement circuit 510 may be configured to measure the impedance value Z_{Inner} of the electrical stimulator 110 and the impedance value Z_{Electrode} of the electrode assembly 120. According to an embodiment of the disclosure, the impedance compensation device 500 (or the measurement circuit 510) may also include the related circuit structure shown in FIG. 4.

According to an embodiment of the disclosure, when the measurement circuit 510 is to measure the non-implantable electrical stimulation device 100 shown in FIG. 1A, FIG. 1B and FIG. 1C, the measurement circuit 510 may provide a high-frequency environment. The frequency is the same as the frequency of the electrical stimulation signal for electrical stimulation of the target area. Herein, the frequency is taken 500 kHz as an example. Then, the measurement circuit 510 may measure the resistance value R_{Electrode}, the capacitance value C_{Electrode}, and the inductance value L_{Electrode} of the electrode assembly 120, and calculate the impedance value Z_{Electrode} of the electrode assembly 120 under the high-frequency signal according to at least one of the measured resistance value R_{Electrode}, capacitance value C_{Electrode}, or inductance value L_{Electrode}. Furthermore, the measurement circuit 510 may measure the resistance value R_{Inner}, the capacitance value C_{Inner}, and the inductance value L_{Inner} of the electrical stimulator 110, and calculate the impedance value Z_{Inner} of the electrical stimulator 110 according to at least one of the measured resistance value R_{Inner}, capacitance value C_{Inner}, or inductance value L_{Inner}. In an embodiment of the disclosure, the inductance value L_{Inner} of the electrical stimulator 110 is not measured. The measurement circuit 510 may write the calculated impedance value Z_{Electrode} of the electrode assembly 120 and the calculated impedance value Z_{Inner} of the electrical stimulator 110 into the firmware of the non-implantable electrical stimulation device 100.

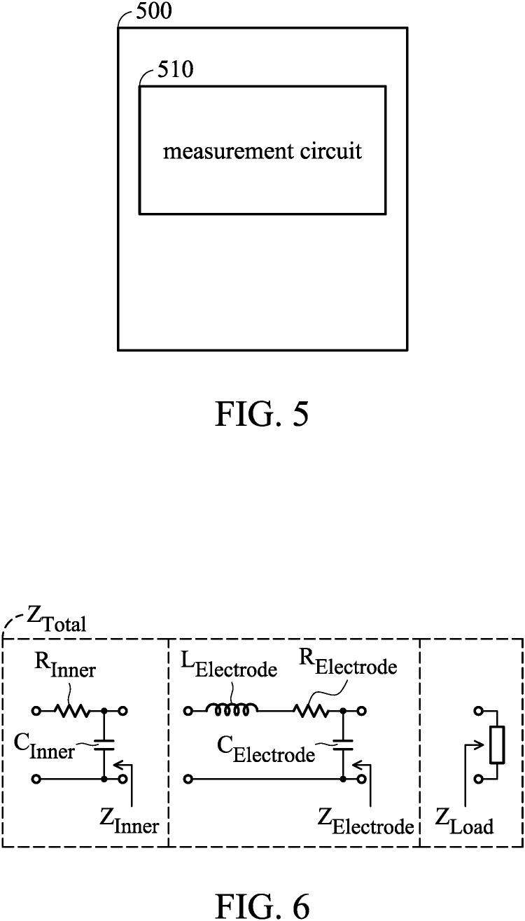

When the non-implantable electrical stimulation device 100 is to calculate the tissue impedance value Z_{Load} of the target area, the non-implantable electrical stimulation device 100 may deduct the impedance value Z_{Electrode} of the electrode assembly 120 and the impedance value Z_{Inner} of the electrical stimulator 110 from the measured total impedance value Z_{Total}, so as to obtain the tissue impedance value Z_{Load} of the target area, as per the impedance compensation model shown in FIG. 6, Z_{Load}=Z_{Total}-Z_{Inner-}Z_{Electrode}, but the disclosure is not limited thereto. In an embodiment of the disclosure, the total impedance value Z_{Total} may be calculated according to the current measured by the current measurement circuit 231 and the voltage measured by the voltage measurement circuit 232 (i.e., R=V/I). Since the calculation manner of the impedance value Z_{Electrode} of the electrode assembly 120 and the impedance value Z_{Inner} of the electrical stimulator 110 may refer to Z= R+j (XL - XC), wherein R is the resistance, XL is the inductive reactance and XC is the capacitive reactance, Z= R+j (XL - XC) is well known to those skilled in the art, and the description thereof is not repeated herein.

According to an embodiment of the disclosure, the measurement circuit 510 may simulate a high-frequency environment according to an electrical stimulation frequency used by the non-implantable electrical stimulation device 100. According to an embodiment of the disclosure, the pulse frequency range of the high-frequency environment provided by the measurement circuit 510 may be in the range of 1 KHz to 1000 KHz. According to an embodiment of the disclosure, the pulse frequency of the high-frequency environment provided by the measurement circuit 510 is the same as that of the electrical stimulation signal.

According to an embodiment of the disclosure, the impedance compensation device 500 may be configured in the external control device 200. According to another embodiment of the disclosure, the impedance compensation device 500 may be configured in the non-implantable electrical stimulation device 100. That is, the high-frequency environment may be provided by the non-implantable electrical stimulation device 100 or the external control device 200. Furthermore, according to another embodiment of the disclosure, the impedance compensation device 500 may also be a stand-alone device (i.e., an impedance analyzer).

According to an embodiment of the disclosure, the impedance compensation device 500 may be applied before the non-implantable electrical stimulation device 100 is produced (i.e., in the laboratory or factory). In an embodiment, before the non-implantable electrical stimulation device 100 is produced, the impedance compensation device 500 may firstly calculate the impedance value Z_{Electrode} of the electrode assembly 120 and the impedance value Z_{Inner} of the electrical stimulator 110, and then write the calculated impedance value Z_{Electrode} of the electrode assembly 120 and the calculated impedance value Z_{Inner} of the electrical stimulator 110 into the firmware of the non-implantable electrical stimulation device 100. It should be noted that the impedance value Z_{Electrode} of the electrode assembly 120 is the overall impedance value of the body 121, the two electrodes 122, the at least one second magnetic unit 123, the at least two electrical connectors 124, and the conductive gel 125.

According to an embodiment of the disclosure, in the electrical stimulation phase and the non-electrical stimulation phase, the impedance compensation device 500 may also perform real-time compensation, i.e., Z_{Inner} and Z_{Electrode} may be measured and obtained every time an electrical stimulation signal is sent. According to an embodiment of the disclosure, the non-electrical stimulation phase refers to in a situation in which the electrical stimulation system 100 and the external control device 200 are just powered on and connected, or after the electrical stimulation device 100 and the external control device 200 are connected, the user has not started the synchronization process of electrical stimulation, or the electrical stimulation device 100 has been attached to the skin of the user and powered on, but the course of providing electrical stimulation has not yet started. The electrical stimulation phase refers to a situation in which the electrical stimulation device 100 has started to provide the course of electrical stimulation.

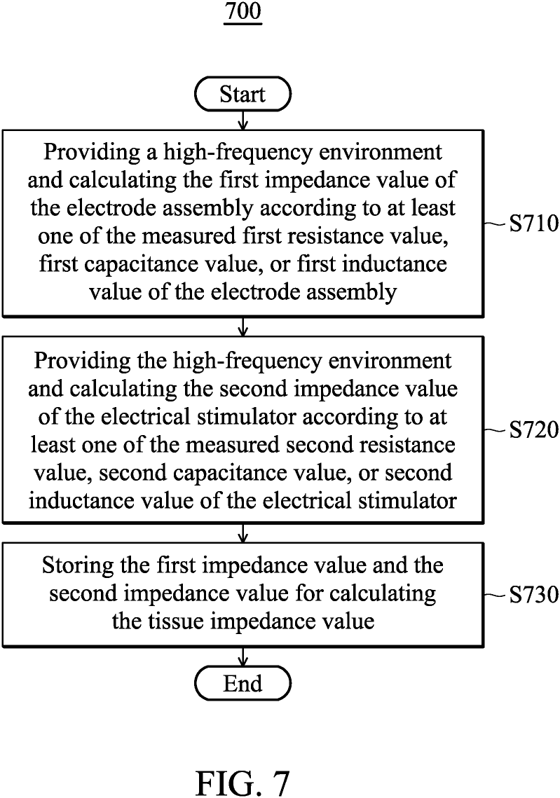

FIG. 7 is a flowchart 700 of an electrical stimulation method for impedance compensation according to an embodiment of the disclosure. flowchart 700 of an electrical stimulation method for impedance compensation is applied to a non-implantable electrical stimulation device 100 for providing high-frequency electrical stimulation, wherein the non-implantable electrical stimulation device 100 includes an electrical stimulator 110 and an electrode assembly 120, and the electrical stimulator 110 is detachably electrically connected to the electrode assembly 120. As shown in FIG. 7, in step S710, the method involves providing a high-frequency environment and calculating the first impedance value of the electrode assembly according to at least one of the measured first resistance value, first capacitance value, or first inductance value of the electrode assembly.

In step S720, the method involves providing the high-frequency environment and calculating the second impedance value of the electrical stimulator according to at least one of the measured second resistance value, second capacitance value, or second inductance value of the electrical stimulator.

In step S730, the method involves storing the first impedance value and the second impedance value for calculating the tissue impedance value.

According to an embodiment of the disclosure, in the above electrical stimulation method for impedance compensation, the non-implantable electrical stimulation device 100 may measure the total impedance value, and deduct the first impedance value of the electrode assembly 120 and the second impedance value of the electrical stimulator 110 from the total impedance value to obtain the tissue impedance value.

According to the electrical stimulation method for impedance compensation provided by the disclosure, when the electrical stimulation device calculates the tissue impedance value, the electrical stimulation device may refer to the pre-calculated impedance value of the electrode assembly and the pre-calculated impedance value of the electrical stimulator to calculate the tissue impedance value, so as to compensate the possible error in the calculation of the tissue impedance value. Therefore, according to the electrical stimulation method for impedance compensation provided by the disclosure, the non-implantable electrical stimulation device (the non-implantable electrical stimulation system) may obtain a more accurate tissue impedance value, which may then be used to calculate the energy value of the electrical stimulation signal transmitted to the target area. That is, the tissue impedance value obtained by using the electrical stimulation method for impedance compensation and the non-implantable electrical stimulation device may then be used to calculate the energy value of the electrical stimulation signal transmitted to the target area.

The serial numbers in the specification and claim, such as “first”, “second”, etc., are only for the convenience of description, and there is no sequential relationship between them.

The steps of the method and the algorithm described in connection with the aspects disclosed herein may be embodied directly in hardware, in a software module executed by a processor, or in a combination of the two. A software module (e.g., including executable instructions and related data) and other data may reside in a data memory such as a random access memory RAM), a flash memory, a read-only memory (ROM), an erasable programmable read only memory (EPROM), an electrically erasable programmable read only memory (EEPROM), a register, a hard disk, a removable disk, a compact disc read only memory (CD-ROM), or any other form of computer-readable storage medium known in the art. A sample storage medium may be coupled to a machine such as, for example, a computer/processor (which may be referred to herein, for convenience, as a “processor”) such that the processor can read information (e.g., code) from and write information to the storage medium. A sample storage medium may be integral to the processor. The processor and the storage medium may reside in an application specific integrated circuit (ASIC). The ASIC may reside in user equipment. Alternatively, the processor and the storage medium may reside as discrete components in user equipment. Moreover, in some aspects any suitable computer-program product may include a computer-readable medium comprising codes relating to one or more of the aspects of the disclosure. In some aspects, a computer program product may include packaging materials.

The above paragraphs describe many aspects. Obviously, the teaching of the disclosure can be accomplished by many methods, and any specific configurations or functions in the disclosed embodiments only present a representative condition. Those who are skilled in this technology will understand that all of the disclosed aspects in the disclosure can be applied independently or be incorporated.

While the disclosure has been described by way of example and in terms of the preferred embodiments, it should be understood that the disclosure is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An electrical stimulation method for impedance compensation, applied to a non-implantable electrical stimulation device for providing high-frequency electrical stimulation, wherein the non-implantable electrical stimulation device comprises an electrical stimulator and an electrode assembly, the electrical stimulator is detachably electrically connected to the electrode assembly, and the electrical stimulation method for impedance compensation comprises:
providing a high-frequency environment and calculating a first impedance value of the electrode assembly according to at least one of the measured first resistance value, first capacitance value, or first inductance value of the electrode assembly;
providing the high-frequency environment and calculating a second impedance value of the electrical stimulator according to at least one of the measured second resistance value, second capacitance value, or second inductance value of the electrical stimulator; and
storing the first impedance value and the second impedance value for calculating a tissue impedance value.

2. The electrical stimulation method for impedance compensation as claimed in claim 1, further comprising:
measuring a total impedance value; and
deducting the first impedance value and the second impedance value from the total impedance value to obtain the tissue impedance value.

3. The electrical stimulation method for impedance compensation as claimed in claim 1, wherein the high-frequency environment is simulated according to an electrical stimulation frequency used by the electrical stimulator.

4. The electrical stimulation method for impedance compensation as claimed in claim 1, wherein the high-frequency environment is a pulse frequency in a range of 1 KHz to 1000 KHz.

5. The electrical stimulation method for impedance compensation as claimed in claim 1, wherein the electrode assembly comprises two electrodes.

6. The electrical stimulation method for impedance compensation as claimed in claim 5, wherein the two electrodes are thin film electrodes.

7. The electrical stimulation method for impedance compensation as claimed in claim 1, wherein the electrical stimulator comprises at least one first magnetic unit, the electrode assembly comprises at least one second magnetic unit, and the electrode assembly is detachably positioned on one side of the electrical stimulator by being attracted by the at least one first magnetic unit and the at least one second magnetic unit.

8. The electrical stimulation method for impedance compensation as claimed in claim 1, wherein the electrode assembly comprises a conductive gel.

9. A non-implantable electrical stimulation system, applied to a high-frequency electrical stimulation operation, comprising:
an electrode assembly;
an electrical stimulator, wherein the electrical stimulator is detachably electrically connected to the electrode assembly; and
an impedance compensation device, configured to provide a high-frequency environment and calculate a first impedance value of the electrode assembly according to at least one of the measured first resistance value, first capacitance value, or first inductance value of the electrode assembly, and provide the high-frequency environment and calculate a second impedance value of the electrical stimulator according to at least one of the measured second resistance value, second capacitance value, or second inductance value of the electrical stimulator;
wherein the first impedance value and the second impedance value are stored in the electrical stimulator for calculating a tissue impedance value.

10. The non-implantable electrical stimulation system as claimed in claim 9, wherein the electrical stimulator measures a total impedance value, and deducts the first impedance value and the second impedance value from the total impedance value to obtain the tissue impedance value.

11. The non-implantable electrical stimulation system as claimed in claim 9, wherein the impedance compensation device simulates the high-frequency environment according to an electrical stimulation frequency used by the electrical stimulator.

12. The non-implantable electrical stimulation system as claimed in claim 9, wherein the high-frequency environment is a pulse frequency in a range of 1 KHz to 1000 KHz.

13. The non-implantable electrical stimulation system as claimed in claim 9, wherein the impedance compensation device is an external control device or is disposed in the electrical stimulator.

14. The non-implantable electrical stimulation system as claimed in claim 9, wherein the electrode assembly comprises two electrodes.

15. The non-implantable electrical stimulation system as claimed in claim 14, wherein the two electrodes are thin film electrodes.

16. The non-implantable electrical stimulation system as claimed in claim 9, wherein the electrical stimulator comprises at least one first magnetic unit, the electrode assembly comprises at least one second magnetic unit, and the electrode assembly is detachably positioned on one side of the electrical stimulator by being attracted by the at least one first magnetic unit and the at least one second magnetic unit.

17. The non-implantable electrical stimulation system as claimed in claim 9, wherein the electrode assembly comprises a conductive gel.
